(19) United States
(12) Patent Application Publication
(10) Pub. No.: US 2014/0171718 A1
(43) Pub. Date: Jun. 19, 2014

(54) METHODS AND APPARATUSES FOR PROVIDING PROCESS CONTROL IN DUAL COLUMN PROCESSES

(71) Applicant: UOP LLC, Des Plaines, IL (US)

(72) Inventors: James W. Harris, Palatine, IL (US); Lewis H. Pettengill, Des Plaines, IL (US)

(73) Assignee: UOP LLC, Des Plaines, IL (US)

(21) Appl. No.: 13/715,827

(22) Filed: Dec. 14, 2012

(51) Int. Cl. C07C 7/00; C07C 7/12

(52) U.S. Cl. CPC C07C 7/005; C07C 7/12
USPC 585/802; 585/831; 210/137

(57) ABSTRACT

Methods and apparatuses for providing process control in dual column processes are provided. In an embodiment, a method for processing a raffinate stream includes forming the raffinate stream in an adsorbent zone. The method monitors the pressure in the adsorbent zone. The raffinate stream is split into a first portion and a second portion. A first flow rate of the first portion is adjusted in response to the pressure in the adsorbent zone, and a second flow rate of the second portion is adjusted in response to the first flow rate. The first portion is fractionated in a first column and the second portion is fractionated in a second column.

### TECHNICAL FIELD

The technical field generally relates to a continuous adsorptive separation process used to separate chemical compounds such as petrochemicals, and more particularly relates to methods and apparatuses that provide process control in dual column processes.

### BACKGROUND

In many commercially important petrochemical and petroleum refining processes it is desired to separate closely boiling chemical compounds or to perform a separation of chemical compounds by structural class. It is normally very difficult or impossible to do this by conventional fractional distillation due to the requirement of numerous columns or excessive amounts of energy. The relevant industries have responded to this problem by utilizing other separation processes which are capable of performing a separation based upon chemical structure or physical characteristics. Adsorptive separation is such a method and is widely used to perform these separations.

In the practice of adsorptive separation, a feed mixture comprising two or more compounds of different skeletal structure or type, such as two isomers, is passed through one or more beds of an adsorbent which selectively adsorbs one compound while permitting the other components of the feed stream to pass through the adsorption zone in an unchanged condition. When the adsorbent reaches a desired loading, the flow of the feed through the adsorbent bed is stopped and the adsorption zone is then flushed to remove non-adsorbed materials surrounding the adsorbent. Thereafter, the desired compound is desorbed from the adsorbent by passing a desorbent stream through the adsorbent bed. The desorbent material is commonly also used to flush non-adsorbed materials from the void spaces around and within the adsorbent prior to performing the actual desorption step. This sequence can be performed in a single large bed of adsorbent or in several parallel beds on a swing bed basis. However, it has been found that in a commercial setting simulated moving bed adsorptive separation provides several important advantages such as high purity and recovery. Therefore, many commercial scale petrochemical separations especially for specific paraffins and xylenes are performed using simulated countercurrent moving beds.

Several economic advantages are derived from the continuous, as compared to batch-wise, operation of a large scale adsorptive separation processes. Recognition of this has driven the development of the simulated moving bed adsorptive separation processes. These processes typically employ a rotary valve and a plurality of lines to simulate the countercurrent movement of an adsorbent bed through adsorption and desorption zones.

The use of a simulated moving bed process to recover para-xylene, ortho-xylene and meta-xylene from mixtures containing other C8 aromatic hydrocarbons is well-known. As the use of simulated moving bed processes has expanded, various improvements have increased capacity. For example, the adsorbent properties within the simulated moving bed have improved dramatically. As a result, unit capacity may be limited by raffinate columns designed for lower raffinate flow rates than currently outputted by improved simulated moving bed processes.

It is contemplated herein that dual raffinate columns be utilized to increase the raffinate column capacity. However, adding a second raffinate column to a simulated moving bed process presents challenges in process control. For example, it is difficult to control the pressure of the chambers in the liquid full environment of the simulated moving bed and raffinate separation processes. Distribution to two raffinate fractionation columns further adds to the complexity of the process control.

Accordingly, it is desirable to provide methods and apparatuses for processing hydrocarbons in simulated moving bed processes. Further, it is desirable to provide process control in dual column processes. It is also desirable to provide methods and apparatuses for fractionating a raffinate stream in dual fractionation columns with feed to one column under pressure control and feed to the other column under flow control. Also, it is desirable to provide such methods and apparatuses that operate economically. Furthermore, other desirable features and characteristics will become apparent from the subsequent detailed description and the appended claims, taken in conjunction with the accompanying drawing and the foregoing technical field and background.

### BRIEF SUMMARY

Methods and apparatuses for providing process control in dual column processes are provided. In an embodiment, a method for processing a raffinate stream includes forming the raffinate stream in an adsorbent zone. The method monitors the pressure in the adsorbent zone. The raffinate stream is split into a first portion and a second portion. A first flow rate of the first portion is adjusted in response to the pressure in the adsorbent zone, and a second flow rate of the second portion is adjusted in response to the first flow rate. The first portion is fractionated in a first column and the second portion is fractionated in a second column.

In another embodiment, a method for processing hydrocarbons is provided. The method includes passing a feed stream containing a first compound and a second compound to a simulated moving bed adsorptive separation process unit. The first compound from the feed stream is adsorbed in the simulated moving bed adsorptive separation process unit and a raffinate stream containing the second compound is formed. The method fractionates a first portion of the raffinate stream in a first raffinate column and a second portion of the raffinate stream in a second raffinate column. Pressure in the simulated moving bed adsorptive separation process unit is monitored to obtain a monitored pressure. A first flow rate of the first portion of the raffinate to the first raffinate column is adjusted in response to the monitored pressure. Further, a second flow rate of the second portion of the raffinate to the second raffinate column is adjusted based on the first flow rate.

In another embodiment, an apparatus for processing hydrocarbons is provided. The apparatus includes an adsorption column containing a bed of adsorbent configured to adsorb a first compound from a feed stream containing the first compound and a second compound. The apparatus further includes a rotary valve configured to selectively pass to the adsorption column the feed stream and a desorbent stream and configured to selectively receive from the adsorption column a raffinate stream and an extract stream containing the first compound. Also, the apparatus includes an extract column configured to recover the first compound from the extract stream, a first raffinate column configured to receive a first portion of the raffinate stream, and a second raffinate column configured to receive a second portion of the raffinate stream. A pressure controller is in communication with the adsorption column to monitor a pressure therein to obtain a monitored pressure. Further, the apparatus includes means for adjusting a first flow rate of the first portion of the raffinate stream to the first raffinate column based on the monitored pressure and means for adjusting a second flow rate of the second portion of the raffinate stream to the second raffinate column based on the first flow rate.

### BRIEF DESCRIPTION OF THE DRAWING

Embodiments of methods and apparatuses for providing process control in dual column processes will hereinafter be described in conjunction with the following drawing figure wherein:

FIG. 1 is a schematic diagram of an embodiment of an apparatus and method for providing process control in dual column processes in accordance with an embodiment.

### DETAILED DESCRIPTION

The following detailed description is merely exemplary in nature and is not intended to limit the methods or apparatuses for providing process control in dual column processes. Furthermore, there is no intention to be bound by any theory presented in the preceding background or the following detailed description.

Methods and apparatuses for processing hydrocarbon streams, and more particularly, for providing pressure control and flow control in dual column arrangements are provided herein. The methods and apparatuses enable the use of dual fractionation columns for processing raffinate streams from adsorbent/separation processes, such as simulated moving bed processes.

In hydrocarbon processing, it is often desired to separate closely boiling chemical compounds or to perform a separation of chemical compounds by structural class. Examples of this are the recovery of normal paraffins from petroleum kerosene fractions for use in the production of detergents and the recovery of para-xylene from a mixture of C8 aromatics with the para-xylene being used in the production of polyesters and other plastics. Meta-xylene is also recovered by adsorptive separation from xylene feed mixtures. The separation of high octane hydrocarbons from a naphtha boiling range petroleum fraction and the recovery of olefins from a mixture of paraffins and olefins are other examples of situations in which the close volatility of the compounds makes the use of fractional distillation impractical. Adsorptive separation of different classes or types of compounds is performed using adsorptive separation when there is an overlap in boiling points across a broad boiling range of compounds. For instance, in the case of the recovery of normal paraffins referred to above it is often desired to recover paraffins having a range of carbon numbers extending from about C9 to C12. This would require at least one fractional distillation column for each carbon number. The resulting capital and operating costs make separation by fractional distillation economically unfeasible.

Other separation methods which are capable of performing a separation based upon chemical structure or characteristics have been identified. Adsorptive separation is often the method of choice and is widely used to perform the separations mentioned above. In adsorptive separation one or more compounds are selectively retained upon an adsorbent and then released by the application of a driving force for the desorption step. The driving force may be heat or a reduced pressure. In the process contemplated herein this driving force is provided by contacting the loaded adsorbent with a desorbent compound. Therefore the adsorbent must be continuously cycled between exposure to the feed stream and a stream comprising the desorbent. As described below this forms at least two effluent streams: the raffinate stream which contains non-adsorbed compounds and the extract stream containing the desired adsorbed compounds. Both streams also comprise the desorbent compound. It is necessary to remove the desorbent from these streams to purify them and also to recover the desorbent for re-use.

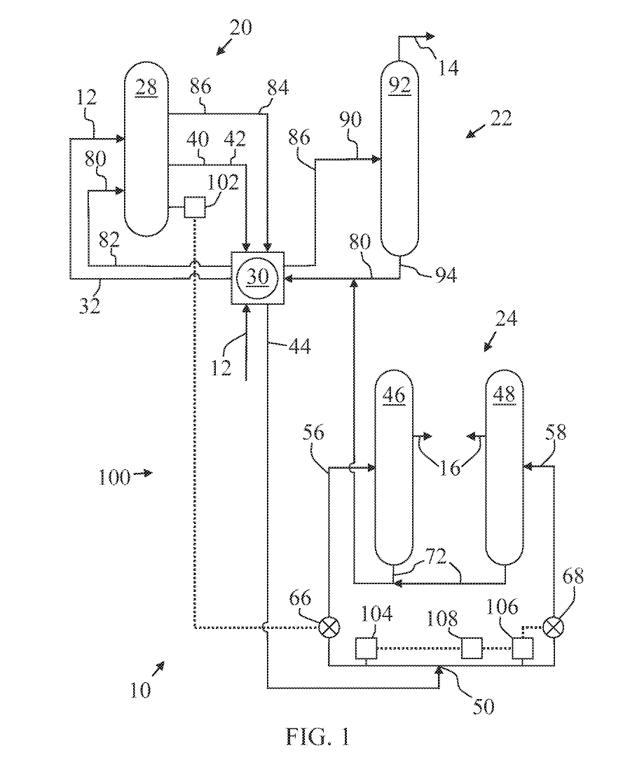

The overall operation of the method and apparatus for processing hydrocarbons described herein may be discerned by reference to FIG. 1. FIG. 1 illustrates an exemplary apparatus 10 for performing a simulated moving bed adsorptive separation process. As shown, the apparatus 10 receives and processes a hydrocarbon stream 12 or feedstock to form an extract product stream 14 and raffinate product streams 16. In an exemplary embodiment, the hydrocarbon stream 12 comprises a mixture of several C8 aromatic hydrocarbons including para-xylene, meta-xylene, ortho-xylene and ethylbenzene. The very close volatilities of these compounds make it impractical to separate them on a commercial scale by fractional distillation. Therefore the predominant commercial separation techniques are crystallization and adsorptive separation.

As shown, the apparatus 10 includes an adsorption zone 20, an extract separation zone 22 and raffinate separation zone 24. The adsorption zone 20 includes a single adsorbent chamber 28 that holds a bed of adsorbent. The method herein is not limited to use with any particular form of adsorbent. The adsorbents employed in the process preferably comprise an inorganic oxide molecular sieve such as a type A, X or Y zeolite or silicalite. Silicalite is well known in the art as a hydrophobic crystalline silica molecular sieve having intersecting bent-orthogonal channels. A wide number of adsorbents are known and a starting molecular sieve is often treated by ion exchange or steaming or other process to adjust its adsorptive properties.

The active component of the adsorbents is normally used in the form of particle agglomerates having high physical strength and attrition resistance. The agglomerates contain the active adsorptive material dispersed in an amorphous, inorganic matrix or binder, having channels and cavities therein which enable fluid to access the adsorptive material. Methods for forming the crystalline powders into such agglomerates include the addition of an inorganic binder, generally a clay comprising a silicon dioxide and aluminum oxide, to a high purity adsorbent powder in a wet mixture. The binder aids in forming or agglomerating the crystalline particles. The blended clay-adsorbent mixture may be extruded into cylindrical pellets or formed into beads which are subsequently calcined in order to convert the clay to an amorphous binder of considerable mechanical strength. The adsorbent may also be bound into irregular shaped particles formed by spray drying or crushing of larger masses followed by size screening. The adsorbent particles may thus be in the form of extrudates, tablets, spheres or granules having a desired particle range, preferably from about 16 to about 60 mesh (Standard U.S. Mesh) (1.9 mm to 250 microns). Clays of the kaolin type, water permeable organic polymers or silica are generally used as binders. The active molecular sieve component of the adsorbents will ordinarily be in the form of small crystals present in the adsorbent particles in amounts ranging from about 75 to about 98-wt. % of the particle based on volatile-free composition. Volatile-free compositions are generally determined after the adsorbent has been calcined at 900° C. in order to drive off all volatile matter.

The performance of an adsorbent is often greatly influenced by a number of factors not related to its composition such as operating conditions, feed stream composition and the water content of the adsorbent. The optimum adsorbent composition and operating conditions for the process are therefore dependent upon a number of interrelated variables. One such variable is the water content of the adsorbent which is expressed herein in terms of the recognized Loss on Ignition (LOI) test. In the LOI test the volatile matter content of the zeolitic adsorbent is determined by the weight difference obtained before and after drying a sample of the adsorbent at 500° C. under an inert gas purge such as nitrogen for a period of time sufficient to achieve a constant weight. For the subject process it is preferred that the water content of the adsorbent results in an LOI at 900° C. of less than 7.0% and preferably within the range of from 0 to 4.0 wt %. The hydration level of the sieve has traditionally been maintained by the injection of water into the feed or desorbent streams.

In the process depicted in FIG. 1, the hydrocarbon stream 12 is passed into a rotary valve 30. The rotary valve 30 has a number of ports or openings corresponding to the number of adsorption chamber process streams plus the number of “bed lines” for connecting to each sub bed of adsorbent located in the one or more adsorbent chambers used in the process. As the adsorbent chamber(s) may contain from about 8 to about 24 adsorbent sub beds, there are a large number of bed lines involved in the process. For simplicity only those four bed lines in use at the moment in time being depicted are shown in FIG. 1.

The rotary valve 30 directs the hydrocarbon stream 12 into bed line 32 which carries it to the adsorbent chamber 28. The hydrocarbon stream 12 enters into the adsorbent chamber 28 at a boundary between two of the sub beds and is distributed across the cross-section of the chamber. It then flows downward through several sub-beds of adsorbent-containing particles. The quantity of adsorbent in these beds selectively retains one compound or structural class of compound, which in this instance is para-xylene. The other components of the feed stream continue to flow downward and are removed from the adsorbent chamber 28 in the raffinate stream 40 carried by line 42. The raffinate stream 40 will also comprise a varying amount of desorbent compound(s) flushed from the inter-particle void volume and removed from the adsorbent itself This desorbent is present in the bed prior to the adsorption step due to the performance of the desorption step.

The raffinate stream 40 enters the rotary valve 30 and is directed into line 44. Line 44 carries the raffinate stream 40 to the raffinate separation zone 24. As shown, the raffinate separation zone 24 includes dual fractionation columns 46 and 48. The raffinate stream 40 is split at point 50 into a first portion 56 and a second portion 58. Each portion 56 and 58 flows through a respective valve 66 and 68 before being introduced into the respective fractionation column 46 and 48. Each fractionation column 46 and 48 is of typical design, including trays for fractionating the raffinate portions 56 and 58. During operation, the raffinate portions entering the columns are separated, with the less volatile desorbent component(s) moving downward out of the fractionation zone and exiting the columns via bottoms 72. As shown, the desorbent in bottoms 72 may be recycled to the desorbent 80 fed to the rotary valve 30. The raffinate products 16 are removed in side-cut streams and may be passed to a xylene isomerization zone to produce more para-xylene.

As shown, a desorbent stream 80 is passed into the adsorbent chamber 28 via line 82. As the desorbent 80 moves downward through selective adsorbent, it removes para-xylene from the adsorbent in a section of the adsorbent chamber 28 used as the desorption zone. This creates a mixture of para-xylene and desorbent which flows through the section of the adsorbent chamber functioning as the desorption zone. As part of this flow, this mixture may be removed from the bottom of the adsorbent chamber 28 and returned to the top of the chamber via a line (not shown) referred to in the art as the pump around line. The liquid then flows through more sub-beds of adsorbent at the top of the adsorbent chamber 28 and is removed from the adsorbent chamber 28 via line 84 as the extract stream 86. This stream 86 passed into the rotary valve 30. The rotary valve 30 directs the extract stream 86 through line 90 and is delivered into the extract separation zone 22 and, specifically, to extract fractionation column 92. Extract fractionation column 92 contains a number of fractionation trays. The more volatile extract component, primarily para-xylene, moves upward through the fractionation zone and is removed from fractionation column 92 as an extract product 14 in an overhead vapor stream. If present in the feed, toluene will to some extent co-adsorb and be present in the extract product 14. It can be removed downstream in a finishing column. The extract product 14 may be passed through an overhead condenser not shown and then into an overhead receiver (not shown). Liquid collected in the receiver may be divided into a reflux stream and returned to the top of the extract fractionation column 92.

The desorbent compound(s) present in the extract stream 86 is driven downward in the extract fractionation column 92. The desorbent 80 leaves the bottom of the extract fractionation column 92 and is returned to the rotary valve 30 in line 94. Makeup desorbent may be added to the process as needed.

In order to provide sufficient and appropriate process control, the apparatus 10 further includes a process control system 100 includes a pressure controller 102 in communication with the adsorbent chamber 28. As a result, the pressure controller 102 is able to monitor the pressure within the adsorbent chamber 28. The pressure controller 102 is further connected to the first valve 66, as shown. Further, the apparatus 10 includes a flow indicator 104 in communication with the first portion 56 of the raffinate stream 40. The flow indicator 104 is able to monitor a first flow rate of the first portion 56 of the raffinate stream 40. Also, the process control system includes a flow controller 106 that is in communication with the second portion 58 of the raffinate stream 40 and with the second valve 68. The flow controller 106 may monitor a second flow rate of the second portion 58 of the raffinate stream 40. As shown, a ratio calculator 108 is interconnected between the flow indicator 104 and the flow controller 106.

With the process control system 100 provided, the flow rates of the raffinate portions 56 and 58 may be maintained and controlled. Specifically, the pressure controller 102 monitors pressure in the adsorbent chamber 28 to obtain a monitored pressure. Based on the monitored pressure in the adsorbent chamber 28, the pressure controller 102 adjusts the flow rate of the first raffinate portion 56 to the first raffinate column 46 through the first valve 66. Specifically, the pressure controller 102 sends a flow rate signal to the first valve 66 through line 110 which adjusts (opens or closes) the first valve 66.

Flow indicator 104 monitors the flow rate of the first raffinate portion 56. Therefore, upon adjustment of the first valve 66, a flow rate change is detected by the flow indicator 104. The flow indicator 104 communicates a flow rate signal to the ratio calculator 108. The ratio calculator 108 may be programmed to control the flow rate of the second raffinate portion 58 at a set ratio to the flow rate of the first raffinate portion 56. For example, the ratio can be set as desired, for example without limitation from about 1:10 to about 10:1. In an exemplary embodiment, the ratio is set at 60:40.

Upon receiving the flow rate signal from the flow indicator 104, the ratio calculator 108 calculates a set point for the flow rate of the second raffinate portion 58. The ratio calculator 108 communicates a flow rate signal, including the set point, to the flow controller 106. In response, the flow controller 106 adjusts the second valve 68 to change the flow rate of the second raffinate portion 58 to the desired set point.

For example, if the pressure controller 102 discerns a pressure decrease in the adsorbent chamber 28, then the first valve 66 is partially closed to decrease the flow rate of the first raffinate portion 56 to the first raffinate column 46. When the flow indicator detects the decrease in the flow rate of the first raffinate portion 56, the ratio calculator provides a correspond lower set point to the flow controller 106, and the flow controller 106 partially closes the valve 68 to decrease the flow rate of the second raffinate portion 58.

Conversely, when the pressure increases in the adsorbent chamber 28, the flow rate of the first raffinate portion 56 is increased, and the flow rate of the second raffinate portion 58 is correspondingly increased.

As a result, the process control system 100 maintains the flow rate of the first raffinate portion 56 on pressure control and the flow rate of the second raffinate portion 58 on flow control in order to properly distribute the raffinate stream 40 while preserving the pressure control requirements of the simulated moving bed process.

Although the apparatus 10 is illustrated to include the rotary valve 30, adsorbent chamber 28, and columns 46 and 48, and 92, it should be understood that the apparatus can further include other equipment or vessels, such as one or more heaters, compressors, condensers, pumps and additional valves, chambers, reboilers, or separation units.

While the preceding description of FIG. 1 has been in terms of the use of a single component “heavy” (less volatile) desorbent in one specific separation, it is not so limited. The adsorbent (stationary phase) and desorbent (mobile phase) are normally selected as a system for each specific separation. The use of multiple component desorbents is, however, very important in some separations. Sometimes the desorbent is less volatile than the extract and raffinate. For instance, the use of a mixture of a normal paraffin and an isoparaffin, both several carbon numbers lighter than the feed, as a desorbent is commercially practiced in the separation of normal paraffins from a mixture of various other types of hydrocarbons.

Operating conditions for adsorption include, in general, a temperature range of from about 20° C. to about 250° C., with from about 60° C. to about 200° C. often being preferred. Adsorption conditions also preferably include a pressure sufficient to maintain the process fluids in liquid phase; which may be from about atmospheric to 600 psig. Desorption conditions generally include the same temperatures and pressure as used for adsorption conditions. It is generally preferred that a simulated moving bed process be operated with an A:F flow rate through the adsorption zone in the broad range of about 1:1 to 5:1.0 where A is the volume rate of “circulation” of selective pore volume of the molecular sieve and F is the volumetric feed flow rate. The practice of the embodiment disclosed herein requires no significant variation in operating conditions, adsorbent or desorbent composition within the adsorbent chambers. That is the adsorbent preferably remains at the same temperature throughout the process.

Although much of the description herein is set in terms of use of the apparatus and method in a simulated moving bed process, the apparatus and method are applicable to other modes of performing adsorptive separation such as a swing bed system employing one or more separate beds of adsorbent. As used herein the phrase simulated moving bed is intended to refer broadly to the different systems which move the point of feed and desorbent insertion into adsorbent to simulate movement of the adsorbent.

Another variation in the performance of the process as depicted in FIG. 1 is the replacement of the rotary valve used as a desorbent flow control device with a manifold system of valves which becomes more practical as the number of sub-beds of adsorbent decreases.

An important characteristic of an adsorbent is the rate of exchange of the desorbent for the extract component of the feed mixture materials or, in other words, the relative rate of desorption of the extract component. This characteristic relates directly to the amount of desorbent material that must be employed in the process to recover the extract component from the adsorbent. Faster rates of exchange reduce the amount of desorbent material needed to remove the extract component, and therefore, permit a reduction in the operating cost of the process. With faster rates of exchange, less desorbent material has to be pumped through the process and separated from the extract stream for reuse in the process. Exchange rates are often temperature dependent. Ideally, desorbent materials should have a selectivity equal to about 1 or slightly less than 1 with respect to all extract components so that all of the extract components can be desorbed as a class with reasonable flow rates of desorbent material, and so that extract components can later displace desorbent material in a subsequent adsorption step.

In adsorptive separation processes, which are generally operated continuously at substantially constant pressures and a temperature which insures all compounds remain in the liquid phase, the desorbent material must be judiciously selected to satisfy many criteria. First, the desorbent material should displace an extract component from the adsorbent with reasonable mass flow rates without itself being so strongly adsorbed as to unduly prevent an extract component from displacing the desorbent material in a following adsorption cycle. Expressed in terms of the selectivity, it is preferred that the adsorbent be more selective for all of the extract components with respect to a raffinate component than it is for the desorbent material with respect to a raffinate component. Secondly, desorbent materials must be compatible with the particular adsorbent and the particular feed mixture. More specifically, they must not reduce or destroy the capacity of the adsorbent or selectivity of the adsorbent for an extract component with respect to a raffinate component. Additionally, desorbent materials should not chemically react with or cause a chemical reaction of either an extract component or a raffinate component. Both the extract stream and the raffinate stream are typically removed from the adsorbent void volume in admixture with desorbent material and any chemical reaction involving a desorbent material and an extract component or a raffinate component or both would complicate or prevent product recovery. The desorbent should also be easily separated from the extract and raffinate components, as by fractionation. Finally, desorbent materials should be readily available and reasonable in cost. With proper attention to desorbent purity, sieve hydration level and adsorbent selection, the ratio of flow rates of desorbent and feed is often below 1:1.

As used herein, various terms and phrases are defined as follows. An “extract” is a compound or class of compounds that is more selectively adsorbed by the adsorbent while a “raffinate” is a compound or type of compound that is less selectively adsorbed. The term “desorbent material” shall mean generally a material capable of desorbing an extract component from the adsorbent. The term “raffinate stream” means a stream in which a raffinate component is removed from the adsorbent bed after the adsorption of extract compounds. The composition of the raffinate stream can vary from essentially 100% desorbent material to essentially 100% raffinate components. The term “extract stream” means a stream in which an extract material, which has been desorbed by a desorbent material, is removed from the adsorbent bed. The extract stream may be rich in the desired compound or may only contain an increased concentration. The term “rich” is intended to indicate a concentration of the indicated compound or class of compounds greater than 50 mol % and preferably above 75 mol %. The composition of the extract stream can vary from essentially 100% desorbent material to essentially 100% extract components. The terms “extract product” and “raffinate product” mean streams produced by the process containing, respectively, an extract component and a raffinate component in higher concentrations than those found in the extract stream and the raffinate stream withdrawn from the adsorbent chamber.

As described herein, an apparatus and method for providing process control in dual column processes have been provided. In exemplary embodiments, an apparatus and method have been described for feeding a first raffinate column with a raffinate stream under process control and a second raffinate column with a raffinate stream under flow control. The apparatus and method described above are particularly well-suited for the extraction of para-xylene from a hydrocarbon stream formed of C8 aromatics using a simulated moving bed process.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the claimed subject matter in any way. Rather, the foregoing detailed description will provide those skilled in the art with a convenient road map for implementing an exemplary embodiment or embodiments. It being understood that various changes may be made in the function and arrangement of elements described in an exemplary embodiment without departing from the scope set forth in the appended claims.

What is claimed is:

1. A method for processing a raffinate stream comprising:
forming the raffinate stream in an adsorbent zone;
monitoring pressure in the adsorbent zone;
splitting the raffinate stream into a first portion and a second portion, a first flow rate of the first portion being adjusted in response to the pressure in the adsorbent zone, and a second flow rate of the second portion being adjusted in response to the first flow rate;
fractionating the first portion in a first column; and
fractionating the second portion in a second column.

2. The method of claim 1 wherein when the pressure in the adsorbent zone rises, the first flow rate is increased.

3. The method of claim 1 wherein the second flow rate is adjusted by setting the second flow rate at a fixed ratio to the first flow rate.

4. The method of claim 3 wherein the fixed ratio is between 10:1 and 1:10.

5. The method of claim 3 wherein the fixed ratio is about 40:60.

6. The method of claim 1 wherein monitoring pressure in the adsorbent zone comprises monitoring pressure in the adsorbent zone with a pressure controller.

7. The method of claim 6 wherein the first portion flows through a first valve to the first column, and wherein the method further comprises communicating a flow signal from the pressure controller to the first valve.

8. The method of claim 7 further comprising monitoring the first flow rate with a flow indicator.

9. The method of claim 8 wherein the second portion flows through a second valve to the second column, and wherein the method further comprises:
communicating a first flow signal from the flow indicator to a ratio calculator;
providing a set point with the ratio calculator based on the first flow signal; and
communicating a second flow signal from the ratio calculator to the second valve.

10. The method of claim 1 wherein forming the raffinate stream in an adsorbent zone comprises adsorbing a selected compound from a feed stream in an adsorbent chamber to form the raffinate stream.

11. The method of claim 10 wherein adsorbing a selected compound from a feed stream in an adsorbent chamber to form the raffinate stream comprises adsorbing the selected compound from the feed stream in a simulated moving bed process to form the raffinate stream, and wherein the method further comprises removing the raffinate stream from the adsorbent zone through a rotary valve.

12. A method for processing hydrocarbons comprising:
passing a feed stream containing a first compound and a second compound to a simulated moving bed adsorptive separation process unit;
adsorbing the first compound from the feed stream in the simulated moving bed adsorptive separation process unit and forming a raffinate stream containing the second compound;
fractionating a first portion of the raffinate stream in a first raffinate column and a second portion of the raffinate stream in a second raffinate column;
monitoring pressure in the simulated moving bed adsorptive separation process unit to obtain a monitored pressure;
adjusting a first flow rate of the first portion of raffinate to the first raffinate column in response to the monitored pressure; and
adjusting a second flow rate of the second portion of raffinate to the second raffinate column based on the first flow rate.

13. The method of claim 12 wherein when the monitored pressure in the simulated moving bed adsorptive separation process unit rises, adjusting the first flow rate comprises increasing the first flow rate.

14. The method of claim 12 wherein the first portion of raffinate passes through a first valve upstream of the first raffinate column, and wherein adjusting the first flow rate comprises adjusting the first valve.

15. The method of claim 14 wherein monitoring pressure in the simulated moving bed adsorptive separation process unit comprises monitoring pressure in the simulated moving bed adsorptive separation process unit with a pressure controller to obtain the monitored pressure, and wherein adjusting the first flow rate comprises communicating a first flow signal from the pressure controller to the first valve.

16. The method of claim 15 wherein the second portion of raffinate passes through a second valve upstream of the second raffinate column, and wherein adjusting the second flow rate comprises adjusting the second valve.

17. The method of claim 16 further comprising:
monitoring flow of the first portion with a flow indicator to obtain the first flow rate;
communicating a first flow signal including the first flow rate to a ratio calculator;
calculating a set point for the second flow rate based on the first flow rate; and
communicating a second flow signal including the set point from the ratio calculator to the second valve, wherein adjusting the second flow rate comprises adjusting the second valve based on the second flow signal.

18. The method of claim 16 wherein adjusting the second flow rate comprises calculating a set point for the second flow rate based on the first flow rate.

19. The method of claim 12 wherein the first compound is para-xylene, and wherein the method further comprises desorbing the para-xylene from the simulated moving bed adsorptive separation process unit with a desorbent to form an extract stream.

20. An apparatus for processing hydrocarbons comprising:
an adsorption column containing a bed of adsorbent configured to adsorb a first compound from a feed stream containing the first compound and a second compound;
a rotary valve configured to selectively pass to the adsorption column the feed stream and a desorbent stream and configured to selectively receive from the adsorption column a raffinate stream and an extract stream containing the first compound;
an extract column configured to recover the first compound from the extract stream;
a first raffinate column configured to receive a first portion of the raffinate stream;
a second raffinate column configured to receive a second portion of the raffinate stream;
a pressure controller in communication with the adsorption column to monitor a pressure therein to obtain a monitored pressure;
means for adjusting a first flow rate of the first portion of the raffinate stream to the first raffinate column based on the monitored pressure; and
means for adjusting a second flow rate of the second portion of the raffinate stream to the second raffinate column based on the first flow rate.
